(19) United States
(12) Patent Application Publication
(10) Pub. No.: US 2016/0279085 A1
(43) Pub. Date: Sep. 29, 2016

(54) Treatment of Severe Hyperlipidemia

(71) Applicant: CymaBay Therapeutics, Inc., Newark, CA (US)

(72) Inventors: Robert L. Martin, San Ramon, CA (US); Charles A. McWherter, Oakland, CA (US); Patrick J. O'Mara, San Ramon, CA (US)

(73) Assignee: CymaBay Therapeutics, Inc., Newark, CA (US)

(21) Appl. No.: 15/159,531

(22) Filed: May 19, 2016

Related U.S. Application Data
(63) Continuation-in-part of application No. 14/541,835, filed on Nov. 14, 2014.
(60) Provisional application No. 61/906,837, filed on Nov. 20, 2013.
(60) Provisional application No. 61/942,438, filed on Feb. 20, 2014.
(60) Provisional application No. 61/974,816, filed on Apr. 3, 2014.
(60) Provisional application No. 61/974,725, filed on Apr. 3, 2014.
(60) Provisional application No. 61/942,941, filed on Feb. 21, 2014.
(60) Provisional application No. 61/974,785, filed on Apr. 3, 2014.

(51) Int. Cl. A61K 31/192; C07K 16/40; A61K 39/395

(52) U.S. Cl. CPC A61K 31/192; A61K 39/3955; C07K 16/40; C07K 2317/21; C07K 2317/24; C07K 2317/76; A61K 2039/505

(57) ABSTRACT

Treatment of severe hyperlipidemia by administration of (R)-2-(4-((2-ethoxy-3-(4-(trifluoromethyl)phenoxy)propyl)thio)-2-methylphenoxy)acetic acid or a salt thereof in combination with a PCSK9 inhibitor.

### CROSS-REFERENCE TO RELATED APPLICATIONS

This application is a continuation-in-part of U.S. application Ser. No. 14/541,833, filed Nov. 14, 2014, entitled “Treatment of homozygous familial hypercholesterolemia”. Application Ser. No. 14/541,833 claims the priority under 35 USC 119(e) of the following six provisional applications: App. No. 61/906,837, filed Nov. 20, 2013; App. No. 61/942,438, filed Feb. 20, 2014; App. No. 61/974,816, filed Apr. 3, 2014; and App. No. 61/974,725, filed Apr. 3, 2014; each entitled “Treatment of dyslipidemias and related conditions”; and App. No. 61/942,941, filed Feb. 21, 2014; and App. No. 61/974,785, filed Apr. 3, 2014; each entitled “Treatment of homozygous familial hypercholesterolemia”. The disclosures of each of these seven applications are incorporated into this application by reference.

### BACKGROUND OF THE INVENTION

1. Field of the Invention

This invention relates to the treatment of severe hyperlipidemia.

2. Description of the Related Art

Dyslipidemia

Dyslipidemia is the presence of an abnormal amount of lipids (e.g. cholesterol and/or fat) in the blood. in developed countries, most dyslipidemias are hyperlipidemias; that is, an elevation of lipids/lipoproteins in the blood—the term hyperlipidemia is often used to include hyperlipoproteinemia. Hyperlipidemias include hypercholesterolemia (elevated cholesterol) and hyperglyceridemia (elevated glycerides), with hypertriglyceridemia (elevated triglycerides (TGs)) as a subset of hyperglyceridemia: combined hyperlipidemia refers to an elevation of both cholesterol and triglycerides. Hyperlipoproteinemia refers to the presence of elevated lipoproteins, usually low-density lipoproteins (LDL), otherwise known as β-lipoproteins, unless otherwise specified; with hyperchylomicronemia (elevated chylomicrons as a subset of hyperlipoproteinemia. Combined hyperlipidemia (mixed hyperlipidemia) refers to elevated TGs and LDL, Familial or primary(i.e., genetically-caused) hypedipidemias are classified according to the Fredrickson classification, which is based on the pattern of lipoproteins on electrophoresis or ultracentrifugation: Type II includes familial hypercholesterolemia (FH, Type IIa) and familial combined hyperlipidemia (Type IIb). Hyperlipidemias such as hypercholesterolemia, combined hyperlipidemia, and hypedipoproteinemia generally involve elevated LDL and low-density lipoprotein cholesterol (LDL-C, “bad cholesterol”), and are frequently accompanied by decreased high density lipoproteins (HDL) and high-density lipoprotein cholesterol (HDL-C, “good cholesterol”). Chronic hyperlipidemia is recognized to be associated with increased risk of atherosclerotic cardiovascular disease and its associated consequences including acute coronary syndrome, myocardial infarction, heart failure, stroke and death.

Familial hypercholesterolemia (FH)

FH is a genetic disorder characterized by high cholesterol levels, specifically very high levels of LDL-C, in the blood, and a high incidence of cardiovascular disease (CVD) at a young age. The high cholesterol levels in FH are less responsive to the kinds of cholesterol control methods that are usually more effective in people without FH (such as dietary modification and statins), because the body's underlying biochemistry is slightly different in these genetically-linked conditions and the body is often overwhelmed by the magnitude of the abnormal levels of lipids. Nevertheless, treatment (including higher statin doses) can often provide benefit, although with effects that are suboptimal. Many patients with FH have mutations in the LDLR gene that encodes the LDL receptor protein, which normally removes LDL from the circulation, or apolipoprotein B (apoB), which is the part of LDL that binds with the receptor, both types of mutations leading to elevated LDL-C; mutations in other genes that affect LDL receptor function do occur, but are less frequent. Patients who have one abnormal copy (heterozygous) of the LDLR gene may have premature CVD at the age of 30 to 40. Patients who have two abnormal copies (homozygous) may experience severe CVD in childhood, and without treatment may experience myocardial infarction, ischemic stroke, and death by around the age of 30. Heterozygous FH (HeFH) is a common genetic disorder, inherited in an autosomal dominant pattern, occurring in 1 in 500 people in most countries; homozygous FH (HoFH) is much rarer, occurring in 1 in 1,000,000 people. HeFH is normally treated with statins, cholesterol absorption inhibitors such as ezetimibe, bile acid sequestrants, or other hypolipidemic agents that lower cholesterol levels, New cases are generally offered genetic counseling, HoFH and the more severe forms of HeFH often do not adequately respond to medical therapy and may require other treatments, including LDL apheresis (removal of LDL in a method similar to dialysis) and, for HoFH, occasionally liver transplantation. Therapies such as statins work primarily by up-regulating liver LDL receptor expression, thereby increasing LDL receptor-mediated clearance of lipids. Thus patients with HoFH (and severe HeFH), who lack functional LDL receptor activity, will generally respond poorly to such therapies. Subjects with receptor-defective HoFH have some residual LDL receptor activity and may see modest reductions in LDL-C with maximal conventional therapy; while subjects with receptor-negative HoFH will generally not benefit significantly. According to Moorjani et al., “Mutations of low-density-lipoprotein-receptor gene, variation in plasma cholesterol, and expression of coronary heart disease in homozygous familial hypercholesterolemia”, Lancet , 341(8856), 1303-1306 (1993), and Goldstein et al, “The LDL Receptor”, Arterioscler. Thromb. Vasc. Biol., 29, 431-438 (2009), patients with receptor-negative HoFH have higher levels of LDL-C (often >750 mg/dL) and develop severe CVD at an earlier age than patients with receptor-defective HoFH (LDL-C levels 400-600 mg/dL). According to Winters, “Low-density lipoprotein apheresis: principles and indications”, Sem Dialysis, 25(2), 145-151 (2012), apheresis reduces CVD events in patients with HoFH. Considerable evidence in other hypercholesterolemic conditions supports the causality of elevated LDL-C in atherosclerotic CVD and the link between lowering LDL-C and reduction in CVD events; so that reductions in LDL-C can be expected to reduce the risk of CVD in HoFH patients.

“Severe hyperlipidemia” refers to HoFH and HeFH; and also to hyperbetalipoproteinemia or combined primary hyperlipidemia in which the person having the condition fails to achieve adequate control of LDL-C with maximally-tolerated conventional lipid-lowering therapy (dietary modification, apheresis if indicated, and one or more of a statin, a cholesterol absorption inhibitor, and a bile acid sequestrant; but not including therapy with a PCSK9 inhibitor). It refers especially to one of these conditions in which the person fails to achieve adequate control of LDL-C with maximally-tolerated conventional lipid-lowering therapy (as above) and therapy with a PCSK9 inhibitor; and also especially to one of these conditions in which the person exhibits symptoms of clinical atherosclerotic CVD. According to Grundy et al., “Implications of recent clinical trials for the National Cholesterol Education Program Adult Treatment Panel III guidelines”, Circulation, 110, 227-239 (2004), the National Cholesterol Education Program has established cardiovascular risk categories related to treatment goals pertinent to severe hyperlipidemia as <70 mg/dL in patients at very high cardiovascular risk, <100 mg/dL in patients at high risk (10-year risk >20%), <130 mg/dL in patients at moderately high risk (10-year risk 10-20%) and moderate risk (10-year risk <10% but with at least two coronary heart disease risk factors , and <160 mg/dL for lower risk. Further, studies reported in Grundy et al. established that, at levels of LDL-C below around 200 mg/dL, a reduction of 30 mg/dL reduced the relative risk of coronary heart disease by about 30%; and FIG. 2 of Amgen's EMDAC Meeting Briefing Document for Evolocumab (http://www.fda.gov/downloads/advisorycommittees/committeesmeetingmaterials/drugs/endocrinologicandmetabolicdrugsadvisorycommittee/ucm450076.pdf), 10 Jun. 2015, at page 26, shows similar results over a wider range. “Adequate control” of LDL-C refers to achievement of an LDL-C level sufficient to significantly decrease cardiovascular risk, such as achievement of the treatment goals quoted in Grundy et al., or such as achievement of one or more of:
(a) an absolute reduction in LDL-C of at least 40 mg/dL, for example at least 100 mg/dL, such as at least 150 mg/dL;
(b) a final LDL-C of not more than 130 mg/dL, for example not more than 100 mg/dL, such as not more than 70 mg/dL; and
(c) a percentage reduction in LDL-C of at least 15%, for example at least 20%, such as at least 30.

PCSK9 Inhibitors as Treatments for Severe Hyperlipidemia

According to Manolis et al., “Novel Hypolipidemic Agents: Focus on PCSK9 Inhibitors”, Hosp. Chron., 9(1), 3-10 (2014), proprotein convertase subtilisin kexin type 9 (PCSK9), is a protein (serine protease) synthesized and secreted mainly by the liver which binds to hepatic LDL receptors. It regulates plasma LDL-C levels by diverting cell surface LDL receptors to lysosomes for degradation. In so doing, PCSK9 prevents the normal recycling of LDL receptors back to the cell surface. This process results in reduced LDL receptor density, decreased clearance of LDL-C, and, consequently, accumulation of LDL-C in the circulation. Thus, PCSK9 levels tend to correlate directly with LDL-C levels, and this correlation is particularly evident on examination of patient populations with differing degrees of LDL receptor function, including those with HoFH and HeFH (see, for example, Raal et al., “Elevated PCSK9 Levels in Untreated Patients with Heterozygous or Homozygous Familial Hypercholesterolemia and the Response to High-Dose Statin Therapy”, J. Am. Heart Assoc., 2, e000028 (http://jaha.ahajournals.org/content/2/2e000028), especially FIG. 2). In animal models, it is known that mutations that increase PCSK9 activity cause hypercholesterolemia and coronary heart disease (CHD); mutations that inactivate PCSK9 lower LDL levels and reduce CHD. PCSK9 inhibitors are therefore considered attractive therapeutic agents for FH, including HoFH. Among the inhibitors under development are the anti-PCSK9 antibodies (i.e. antibodies that bind to PCSK9 and prevent it binding to liver LDL receptors) evolocumab, alirocumab, bococizumab, RG7652, LY3015014, and LGT-209, of which evolocumab and alirocumab are the furthest advanced; the antisense RNAi oligonucleotide ALN-PCSsc (a GalNAc-modified second generation subcutaneously-administrable agent based on ALN-PCS); the pegylated adnectin BMS-962476; and others.

Evolocumab (REPATHA) has been approved in the United States (August 2015), by the European Medicines Authority (July 2015), and in Canada (September 2015). In the US, evolocumab is indicated as an adjunct to diet and: (a) maximally tolerated statin therapy for treatment of adults with HeFH or clinical atherosclerotic CVD, who require additional lowering of LDL-C; and (b) other LDL-lowering therapies (e.g., statins, cholesterol absorption inhibitors, LDL apheresis) in patients with HoFH who require additional lowering of LDL-C. The approved US dosing is 140 mg every 2 weeks or 420 mg once monthly subcutaneously for hyperlipidemia/HeFH and 420 mg once monthly subcutaneously for HoFH (though 420 mg every 2 weeks subcutaneously has also been tested and proved more effective). Approval was based on data from the PROFICIO program, in which evolocumab reduced LDL-C levels in hypercholesterolemic subjects more than 50%. Evolocumab has also been tested in 331 HeFH patients in the RUTHERFORD-2 trial (Raal et al., “PCSK9 inhibition with evolocumab (AMG 145) in heterozygous familial hypercholesterolaemia (RUTHERFORD-2): a randomised, double-blind, placebo-controlled trial”, Lancet, online publication Oct. 2, 2014), using subcutaneous injection of 140 mg every 2 weeks or 420 mg every month by subcutaneous injection. Significant reductions in LDL-C were seen in both treatment groups relative to placebo; and evolocumab was said to be well tolerated, with the most common AEs occurring more frequently in the treatment groups being nasopharyngitis (9% vs. 5% for placebo) and muscle-related AEs (5% vs. 1%). Evolocumab has also been tested in 49 HoFH patients, using subcutaneous injection of 42.0 mg every month by subcutaneous injection. Significant reductions in LDL-C were seen in the treatment group relative to placebo (baseline average LDL-C was 349 mg/dL, mean change relative to placebo was −31% (−108 mg/dL)). However, the US approved labeling for evolocumab notes that patients who were known to have two LDL-receptor negative alleles did not respond to treatment with evolocumab.

Alirocumab (PRALUENT) has been approved in the United States and by the European Medicines Authority (both in July 2015). In the US, alirocumab is indicated as an adjunct to diet and maximally tolerated statin therapy for treatment of adults with HeFH or clinical atherosclerotic CVD, who require additional lowering of LDL-C. The approved US dosing is 75 mg every 2 weeks subcutaneously, with escalation to 150 mg every 2 weeks subcutaneously if adequate control of LDL-C is not achieved with the lower dose. Alirocumab has also been tested in hypercholesterolemia and in a placebo-controlled Phase 2 study in HeFH using subcutaneous injection at 150 mg every 2 weeks or 150, 200, or 300 mg every 4 weeks, with significant reductions seen in LDL-C (29% for 150 mg/4 weeks to 68% for 150 mg/2 weeks).

Bococizumab has been tested in hypercholesterotemia and is under study in HeFH. A Phase 2 study in hypercholesterolemia using subcutaneous injection at 50, 100, or 150 mg twice monthly or 200 or 300 mg once monthly in 354 patients, with dose lowering if LDL-C was reduced to ≦25 mg/dL, showed significant reductions in LDL-C at week 12, with the greatest reductions seen with 150 mg for the twice monthly regimen and 300 mg for the once monthly regimen. The Phase 3 trials are using every 2 week dosing, at 75 or 150 mg, ALN-PCS completed a single ascending dose Phase 1 study in hypercholesterolemic subjects, using intravenous doses between 0.015 and 0.040 mg/Kg, with a mean 70% reduction in PCSK9 at the highest dose, while ALN-PCS was said to be well tolerated. BMS-962476 has completed a single ascending dose Phase 1 study in hypercholesterolemic subjects, using subcutaneous doses of 0.01, 0.03, 0.1, and 0.3 mg/Kg and intravenous doses of 0.3 and 1.0 mg/Kg alone, and 0.1 and 0.3 mg/Kg in combination with statins. BMS-962476 was said to be well tolerated, and doses ≧0.3 mg/Kg reduced PCSK9 by at least 90%.

PCSK9 has been found to have additional functions beyond those described for its interactions with the LDL receptor. For example, Sun et al., “Proprotein Convertase Subtilisin/Kexin Type 9 Interacts With Apolipoprotein B and Prevents Its Intracellular Degradation, Irrespective of the Low-Density Lipoprotein Receptor”, Arterioscler. Thromb. Vasc. Biol., 32, 1585-1595 (2012), studied the role of PCSK9 in mice in which the LDL receptor gene had been deleted. They discovered that increased PCSK9 increases plasma cholesterol and triglycerides in an LDL receptor independent fashion. Their results indicated that PCSK9 interacts with the core protein apoB to increase the secretion of VLDL, the precursor to circulating LDL. Cameron et al., “Serum levels of proprotein convertase subtilisin/kexin type 9 in subjects with familial hypercholesterolemia indicate that proprotein convertase subtilisin/kexin type 9 is cleared from plasma by low-density lipoprotein receptor—independent pathways”, Translational Res., 160, 125-130 (2012), and Canuel et al., “Proprotein Convertase Subtilisin/Kexin Type 9 (PCSK9) Can Mediate Degradation of the Low Density Lipoprotein Receptor-Related Protein 1 (LRP-1)”, PLoS ONE 8(5), e64145 (2013), have also shown additional functions for PCSK9 independent of the LDL receptor. Their studies implicated roles for PCSK9 with other receptors including the Low Density Receptor Related Protein 1 (LRP-1) which have roles in trafficking of lipoproteins with roles in human health beyond those of LDL.

Patients on PCSK9 therapy often have lowering of LDL-C, and some show dramatic lowering to well below 100 mg/dL, but many do not reach treatment goals, or achieve adequate control of LDL-C, For example, in the Phase 3 clinical study with alirocumab in very high risk patients (Robinson et al., “Efficacy and Safety of Alirocumab in Reducing Lipids and Cardiovascular Events”, N. Engl. J. Med.,372(16), 1489-1499 (2015) (Epub 2015 Mar. 15) approximately 20% of patients failed to achieve the goal of <70 mg/dL. In the TESLA Phase 2/3 studies with evolocumab in HoFH patients (EMDAC Meeting Briefing Document for Evolocumab, Tables 10 and 11, pages 70 and 72) patients with baseline LDL-C of 356 to 442 mg/dL achieved on average a 23% reduction in LDL-C concentration (31% relative to placebo, where patients saw an increase in LDL-C), indicating that most were still not athieving adequate control of LDL-C, or achieving their treatment goals for CVD risk reduction.

MBX-8025

MBX-8025 is the compound of the formula

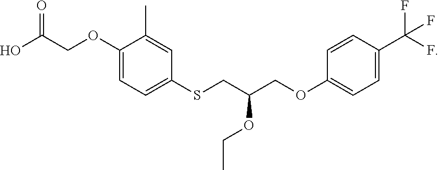 [SMILES: CCO[C@H](COc1ccc(C(F)(F)F)cc1)CSc1ccc(OCC(=O)O)c(C)c1]

MBX-8025 has the chemical name (R)-2-(4-((2-ethoxy-3-(4-(trifluoromethyl)phenoxy)propyl)thio-2-methylphenoxy)acetic acid [IUPAC name as generated by CHEMDRAW ULTRA 12.0]. MBX-8025 and its synthesis, formulation, and use is disclosed in for example, U.S. Pat. No. 7,301,050 (compound 15 in Table 1, Example M, claim 49), U.S. Pat. No. 7,635,718 (compound 15 in Table 1, Example M), and U.S. Pat. No. 8,106,095 (compound 15 in Table 1, Example M, claim 14). Lysine (L-lysine) salts of MBX-8025 and related compounds are disclosed in U.S. Pat. No. 7709682 (MBX-8025 L-lysine salt throughout the Examples, crystalline forms claimed).

MBX-8025 is an orally active, potent (2 nM) agonist of peroxisome proliferator-activated receptor-δ (PPARδ), which is also specific (>600-fold and >2500-fold compared with PPARα and PPARγ receptors). PPARδ activation stimulates fatty acid oxidation and utilization, improves plasma lipid and lipoprotein metabolism, glucose utilization, and mitochondrial respiration, and preserves stem cell homeostasis. According to U.S. Pat. No, 7,301,050, PPARδ agonists, such as MBX-8025, are suggested to treat PPARδ-mediated conditions, including “diabetes, cardiovascular diseases, Metabolic X syndrome, hypercholesterolemia, hypo-HDL-cholesterolemia, hyper-LDL-cholesterolemia, dyslipidernia, atherosclerosis, and obesity”, with dyslipidemia said to include hypertriglyceridemia and mixed hyperlipidemia.

A Phase 2 study of MBX-8025 L-lysine dihydrate salt in mixed dyslipidemia (6 groups, 30 subjects/group: once daily placebo, atorvastatin 20 mg, or MBX-8025 L-lysine dihydrate salt at 50 or 100 mg (calculated as the free acid) capsules alone or combined with atorvastatin 20 mg, for 8 weeks) has been reported by Bays et al., “MBX-8025, A Novel Peroxisome Proliferator Receptor-δ Agonist: Lipid and Other Metabolic Effects in Dyslipidemic Overweight Patients Treated with and without Atorvastatin”, J. Clin. Endoerin. Metab., 96(9), 2889-2897 (2011) and Choi et al., “Effects of the PPAR-δ agonist MBX-8025 on atherogenic dyslipidemia”, Atherosclerosis, 220, 470-476 (2012). Compared to placebo, MBX-8025 alone and in combination with atorvastatin significantly (P<0.05) reduced apoB100 by 20-38%, LDL by 18-43%, triglycerides by 26-30%, non-HDL-C by 18-41%, free fatty acids by 16-28%, and high-sensitivity C-reactive protein by 43-72%; it raised HDL-C by 1-12% and also reduced the number of patients with the metabolic syndrome and a preponderance of small LDL particles. While MBX-8025 at 100 mg/day reduced LDL-C by 22% over the total population treated, the percentage reduction in LDL-C increased to 35% in the tertile with the highest starting LDL-C levels (187-205 mg/dL), and trend analysis on individual patient data confirmed a positive correlation between percentage reduction in LDL-C and starting LDL-C level. MBX-8025 reduced LDL-S/VS by 40-48% compared with a 25% decrease with atorvastatin; and MBX-8025 increased LDL-L by 34-44% compared with a 30% decrease with atorvastatin. MBX-8025 significantly reduced alkaline phosphatase by 32-43%, compared to reductions of only 4% in the control group and 6% in the ATV group; and significantly reduced γ-glutamyl transpeptidase by 24-28%, compared to a reduction of only 3% in the control group and an increase of 2% in the ATV group. Thus MBX-8025 corrects all three lipid abnormalities in mixed dyslipidemia—lowers TGs and LDL and raises HDL, selectively depletes small dense LDL particles (92%), reduces cardiovascular inflammation, and improves other metabolic parameters including reducing serum aminotransferases, increases insulin sensitivity (lowers HOMA-IR, fasting plasma glucose, and insulin), lowersγ-glutamyl transpeptidase and alkaline phosphatase, significantly (>2-fold) reduces the percentage of subjects meeting the criteria for metabolic syndrome, and trends towards a decrease in waist circumference and increase in lean body mass. MBX-8025 was safe and generally well-tolerated, and also reduced liver enzyme levels. As explained in U.S. Patent Application Publication No. 2010-0,152,295, MBX-8025 converts LDL particle size pattern I to pattern A; and from pattern B to pattern I or A, where LDL particle size pattern B is a predominant LDL particle size of less than 25.75 nm, pattern I is a predominant LDL particle size of from 25.75 nm to 26.34 nm, and pattern A is a predominant LDL particle size of greater than 26.34 nm, where the LDL particle size is measured by gradient-gel electrophoresis.

According to Shiomi and Ito, “The Watanabe heritable hyperlipidemic rabbit, its characteristics and history of development: A tribute to the late Dr. Yoshio Watanabe”, Atherosclerosis, 207(1), 1-7 (2009), the Watanabe-heritable hyperlipidemic (WHHL) rabbit is a preclinical model of FH that is characterized by low (<5%) hepatic LDL-R activity, highly elevated LDL-C and the accompanying development of atherosclerosis; and is used in studies of candidate compounds for the treatment of hypercholesterolemia and atherosclerosis. CymaBay Therapeutics has reported (“CymaBay Therapeutics Announces Preclinical Data Demonstrating the Potential of MBX-8025 to Treat Homozygous Familial Hypercholesterolemia”, Jan. 28, 2015, http://ir.cymabay.com/press-releases/detail/244/cymabay-therapeutics-announces-preclinical-data-demonstrating-the-potential-of-mbx-8025-to-treat-homozygous-familial-hypercholesterolemia) a study of MBX-8025 in the WHHL rabbit. In this study, five WHHL rabbits with highly elevated baseline plasma LDL-C levels (360-592 mg/dL) were dosed by subcutaneous administration of MBX-8025 (30 mg/kg) once daily for three weeks, followed by a four-week washout period. LDL-C concentrations were measured once weekly during treatment (weeks 1-3) and after washout of MBX-8025 (week 7). Treatment with MBX-8025 resulted in changes from baseline in mean LDL-C of −33,−45, and −42% at weeks 1, 2 and 3, respectively, All animals experienced absolute decreases in LDL-C (114-302 mg/dL; p<01 for all changes vs. baseline); and the LDL-C lowering effect of MBX-8025 was completely reversed after a washout period of 4 weeks.

CymaBay Therapeutics has also reported (“CymaBay Therapeutics Announces Positive Results from its Pilot Phase 2 Clinical Study of MBX-8025 in Patients with Homozygous Familial Hypercholesterolemia”, Mar. 17, 2016, http://ir.cymabay.com/press-releases/detail/361/cyrnabay-therapeutics-announces-positive-results-from-its-pilot-phase-2-clinical-study-of-mbx-8025-in-patients-with-homozygous-familial-hypercholesterolemia) the results of a pilot Phase 2 clinical study in HoFH patients. The study was an open label, dose escalation study of 12 weeks duration; thirteen patients were enrolled, all of whom had genetically confirmed HoFH, including two subjects who had functionally negative mutations in their LDL receptor (LDL-R) genes. All of the subjects were taking ezetimibe and were on maximum statin therapy (i.e. were receiving maximally-tolerated conventional lipid-lowering therapy). None of the study participants received lomitapide, mipomersen or a PCSK9 inhibitor. Eight patients were undergoing concomitant apheresis on a weekly or biweekly schedule. Despite being on maximal conventional therapy, the average baseline LDL-C was 368 mg/dL. Subjects received once daily treatment with 50 mg of MBX-8025 for 4 weeks, after which the dose was escalated to 100 and 200 mg in successive 4-week periods. Two per-protocol analyses were performed on 12 of the 13 subjects (1 subject was excluded because of multiple missed apheresis visits, resulting in marked fluctuations in LDL-C levels). A responder analysis was carried out which reflects the largest decrease in LDL-C observed during treatment for each subject: 3 subjects had a ≧30% decrease, 5 had a ≧20% decrease, including one LDLR negative subject, 7 had a >15% decrease, and 5 had a <15% decrease; while the average maximum decrease was 19%. Because of the high baseline LDL-C levels in these individuals, these percentage decreases correspond to significant absolute decreases in LDL-C (mean decrease of 109 mg/dL for the subjects with ≧15% decrease), Mean PCSK9 was elevated at baseline (544±133 ng/mL), as anticipated for patients with HoFH, but unexpectedly increased significantly during treatment by a mean of 43% in these patients.

Most treatments are less effective when treating severe hyperlipidemia than they are when treating more common forms of hyperlipidemia; and this has been true for statins, cholesterol absorption inhibitors, PCSK9 inhibitors, and MBX-8025.

The disclosures of the documents referred to in this application are incorporated into this application by reference.

### SUMMARY OF THE INVENTION

This invention is the treatment of severe hyperlipidemia, comprising administration of (R)-2-(4-((2-ethoxy-3-(4-(trifluoromethyl)phenoxy)propyl)thio)-2-methylphenoxy)acetic acid or a salt thereof (MBX-8025 or an MBX-8025 salt) in combination with a PCSK9 inhibitor. In particular, this invention is the treatment of severe hyperlipidemia in persons who fail to achieve adequate control of LDL-C with maximally-tolerated conventional lipid-lowering therapy. It refers especially to one of these conditions in which the person fails to achieve adequate control of LDL-C with maximally-tolerated conventional lipid-lowering therapy and therapy with a PCSK9 inhibitor; and also especially to one of these conditions in which the person exhibits symptoms of clinical atherosclerotic CVD.

In various aspects, this invention includes: methods of treating severe hyperlipidemia by administering MBX-8025 or an MBX-8025 salt in combination with a PCSK9 inhibitor; and kits for treating severe hyperlipidemia comprising compositions comprising MBX-8025 or an MBX-8025 salt, in combination with compositions containing a PCSK9 inhibitor.

The PCSK9 inhibitor may be an anti-PCSK9 antibody such as evolocumab, alirocumab, bococizumab, RG7652, LY3015014, and LGT-209; an antisense RNAi oligonucleotide such as ALN-PCSsc; or an adnectin such as BMS-962476.

Because the effects of MBX-8025, mediated by PPAδ, do not require an effective LDLR to lower LDL-C and improve other lipid parameters (an effect seen in knockout mice lacking LDLR), MBX-8025 will have a special benefit in persons with HoFH. Also, because the effect of MBX-8025 on LDL-C reduction has been seen to increase in dyslipidemic patients with higher starting LDL-C levels, MBX-8025 is expected to be especially effective in severe hyperlipidemia, such as in HoFH, where starting LDL-C levels may be extremely elevated. Finally, because treatment with MBX-8025 has been shown to lower LDL-C despite increasing PCSK9 (an effect that might be expected to increase LDL-C), and because treatment with maximally-tolerated lipid-lowering therapy plus therapy with a PCSK9 inhibitor produces greater lowering of LDL-C (in percentage or absolute terms) than therapy with MBX-8025 alone, the addition of treatment with MBX-8025 to treatment with a PCSK9 inhibitor, where the offsetting effect of increasing PCSK9 will be blocked, is expected to be especially effective.

Preferred embodiments of this invention are characterized by the specification and by the features of Claims 1 to 20 of this application as filed.

### DETAILED DESCRIPTION OF THE INVENTION

Definitions

Dyslipidemia″, including HoFH, HeFH, hyperbetalipoproteinemia, and mixed hyperlipidemia, is described in paragraphs [0003] through [0007]; with “severe hyperlipidemia” described in paragraph [0007]. “Maximally-tolerated conventional lipid-lowering therapy”, and “adequate control” of LDL-C are also described in paragraph [0007].

“PCSK9 inhibitors”, including anti-PCSK9 antibodies such as evolocumab, alirocumab, bococizumab, RG7652, LY3015014, and LGT-209; antisense RNAi oligonucleotides such as ALN-PCSsc; and adnectins such as BMS-962476, are described in paragraphs [0008] through [0014], respectively.

“NIBX-8025” and its salts, are described in paragraphs [0015] through [0020].

A “therapeutically effective amount” of each of (MBX-8025 or an MBX-8025 salt) and a PCSK9 inhibitor means that amount which, when administered in combination therapy to a human for treating severe hyperlipidemia, is sufficient to effect treatment for severe hyperlipidernia. “Treating” or “treatment” of severe hyperlipidemia in a human includes one or more of:

(1) preventing or reducing the risk of developing severe hyperlipidemia, i.e., causing at least one of the clinical symptoms of severe hyperlipidemia not to develop in a subject who may be predisposed to severe hyperlipidemia but who does not yet experience or display symptoms of the severe hyperlipidemia (i.e. prophylaxis);

(2) inhibiting severe hyperlipidemia, i.e., arresting or reducing the development of severe hyperlipidemia or at least one of its clinical symptoms; and

(3) relieving severe hyperlipidemia, i.e., causing regression, reversal, or amelioration of severe hyperlipidemia or reducing the number, frequency, duration or severity of a least one of its clinical signs, symptoms, or consequences.

Desirably, the treatment will be one in which a subject receiving maximally-tolerated conventional lipid-lowering therapy (one or more of a statin, a cholesterol absorption inhibitor, and a bile acid sequestrant) and therapy with a PCSK9 inhibitor exhibits one or more of:

(a) an absolute reduction in LDL-C of at least 40 mg/dL, for example at least 100 mg/dL, such as at least 150 mg/dL;

(b) a final LDL-C of not more than 130 mg/dL for example not more than 100 mg/dL, such as not more than 70 mg/dL; and

(c) a percentage reduction in LDL-C of at least 15%, for example at least 20%, such as at least 30%, when receiving both the maximally-tolerated conventional lipid-lowering therapy and therapy with a PCSK9 inhibitor, and therapy with (MBX-8025 or an MBX-8025 salt).

The therapeutically effective amount for a particular subject varies depending upon the health and physical condition of the subject to be treated, the type and extent of severe hyperlipidemia, the assessment of the medical situation, and other relevant factors. It is expected that the therapeutically effective amount will fall in a relatively broad range, as discussed below, and that this amount can be determined through routine trial based on the ordinary skill in the art and the guidance of this application.

Salts (for example, pharmaceutically acceptable salts) of MBX-8025 and of the PCSK9 inhibitor are included in this invention and are useful in the compositions, methods, and uses described in this application. These salts are preferably formed with pharmaceutically acceptable acids and bases. See, for example, “Handbook of Pharmaceutically Acceptable Salts”, Stahl and Wertnuth, eds., Verlag Helvetica Chimica Acta, Zürich, Switzerland, for an extensive discussion of pharmaceutical salts, their selection, preparation, and use. Unless the context requires otherwise, reference to MBX-8025 and other compounds is a reference both to the compound and to its salts.

Because MBX-8025 contains a carboxyl group, it may form salts when the acidic proton present reacts with inorganic or organic bases. Typically the MBX.-8025 is treated with an excess of an alkaline reagent, such as hydroxide, carbonate or alkoxide, containing an appropriate cation. Cations such as Na⁺, K⁺, Ca²⁺, Mg²⁺, and NH₄⁺are examples of cations present in pharmaceutical acceptable salts. Suitable inorganic bases, therefore, include calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amities including isopropylamine, trimethylamine, diethylamine, triethylamine, tripropylamine, ethanolatnine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. As noted in paragraph [0018], MBX-8025 has been studied in clinical trials as its L-lysine dihydrate salt, and MBX-8025 has also been studied in clinical trials as its calcium salt.

“Combination therapy” with MBX-8025 and a PCSK9 inhibitor means administration of MBX-8025 and a PCSK9 inhibitor during the course of treatment of severe hyperlipidemia. Such combination therapy may involve administration of a PCSK9 inhibitor before, during, and/or after administration of MBX-8025, such that therapeutically effective levels of each of the compounds are maintained. MBX-8025 is typically administered orally once/day. Because the PCSK9 inhibitors are administered by injection less frequently, such as once every 2 or 4 weeks for the PCSK9 antibodies, it may be convenient to administer the PCSK9 inhibitors, on the day selected for their administration, at the same time as the MBX-8025 is administered.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of ”; and, for example, a formulation “comprising” a compound must contain that compound but also may contain other active ingredients and/or excipients:

Formulation and Administration

The MBX-8025, and the PCSK9 inhibitor, may be administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosa membranes or transdermal formulations. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because MBX-8025 is orally available, typical formulations will be oral, and typical dosage forms of MBX-8025 will be tablets or capsules for oral administration. As mentioned in paragraph [0018], MBX-8025 has been formulated in capsules for clinical trials. The PCSK9 inhibitors are all formulated as solutions for injection, typically for subcutaneous injection.

Depending on the intended mode of administration, the pharmaceutical compositions may be in the form of solid, semi-solid or liquid dosage forms, preferably in unit dosage form suitable for single administration of a precise dosage. In addition to an effective amount of the MBX-8025 and the PCSK9 inhibitor, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule, or it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a kit comprising separate compositions of MBX-8025 and of a PCSK9 inhibitor, is packaged in a container with a label, or instructions, or both, indicating use of the kit in the treatment of severe hyperlipidemia.

When MBX-8025 and a PCSK9 inhibitor are used in combination therapy, a suitable amount of MBX-8025 (calculated as the free acid) for oral dosing will be 20-200 mg/day, preferably 50-200 mg/day; and suitable amounts of the PCSK9 inhibitor will be similar to the amounts approved or used in clinical trials, as described in paragraphs [0008] through [0014], such as 140 mg every 2 weeks or 420 mg once monthly subcutaneously for hyperlipidemia/HeFH and 420 mg once monthly or every 2 weeks subcutaneously for HoFH for evolocumab and 75 mg or 150 mg every 2 weeks subcutaneously for alirocumab, That is, suitable amounts of MBX-8025 and the PCSK9 inhibitor to achieve a therapeutically effective amount of the combination therapy will be similar to the amounts employed in clinical trials (and currently approved, in the case of evolocumab and alirocumab). However, it is possible that the therapeutically effective amounts of either may be less in combination therapy than when used as monotherapy because each of MBX-8025 and the PCSK9 inhibitors is useful in lowering cholesterol, particularly LDL-C.

A person of ordinary skill in the art of the treatment of severe hyperlipidemia will be able to ascertain the therapeutically effective amounts of MBX-8025 and a PCSK9 inhibitor, when used in combination therapy, for a particular patient and type and stage of severe hyperlipidemia to achieve a therapeutically effective amount without undue experimentation and in reliance upon personal knowledge and the disclosure of this application.

### EXAMPLES

### Example 1

### Dose Escalation Study with MBX-8025 and Evolocumab in HoFH

Subjects with HoFH (diagnosed either by genetic testing or by an untreated LDL-C >500 mg/dL and early appearance of xanthoma or LDL-C levels consistent with HeFH in both parents), on maximally-tolerated lipid-lowering therapy (one or more of a statin, a cholesterol absorption inhibitor, and a bile acid sequestrant) and evolocumab at 420 mg once monthly, are treated with MBX-8025 L-lysine dihydrate salt at a dose of 50, 100, or 200 mg/day (as MBX-8025 free acid), escalating every 4 weeks. The subjects are instructed to maintain a low-fat diet (<20% energy from fat) and to take dietary supplements that provide approximately 400 IU vitamin E, 210 mg α-linolenic acid, 200 mg linoleic acid, 110 mg eicosapentenoic acid, and 80 mg docosahexaenoic acid per day; and are permitted their usual other medications. The subjects are assessed before the study, and at intervals during the study, such as every 1, 2, and 4 weeks after the start of a new dose and 4 weeks after the last dose of the combination therapy, for safety and pharmacodynamic evaluations. MRIs of the subjects' livers are taken after 4 weeks at each dose, and 4 weeks after study completion, to determine hepatic fat. At each visit, after a 12-hour fast, blood is drawn and urine collected; and a standard metabolic panel, complete blood count, and standard urinalysis are performed. Blood is analyzed for total cholesterol (TC), HDL-C, TG, VLDL-C, LDL-C and apoB. The subjects also maintain health diaries, which are reviewed at each visit.

The combination of MBX-8025 and evolocumab causes dose-dependent lowering of TC, LDL-C, VLDL-C, TG, and apoB, and raising of HDL-C; in particular, increasing the lowering of TC, LDL-C, VLDL-C, TG, and apoB, and raising of HDL-C, beyond that caused by evolocumab alone.

Similar studies may be conducted with MBX-8025 and other PCSK9 inhibitors, such as alirocumab; and an increased reduction in LDL-C over that caused by the PCSK9 inhibitor alone is expectable.

### Example 2

### Dose Escalation Study with MBX-8025 and Evolocumab in HeFH

Subjects with HeFH, on maximally-tolerated lipid-lowering therapy (one or more of a statin, a cholesterol absorption inhibitor, and a bile acid sequestrant) and evolocumab at either 140 mg every 2 weeks or 420 mg once monthly, are treated with MBX-8025 L-lysine dihydrate salt at a dose of 50, 100, or 200 mg/day (as MBX-8025 free acid), escalating every 4 weeks. The subjects are instructed to maintain a low-fat diet (<20% energy from fat) and to take dietary supplements that provide approximately 400 IU vitamin E, 210 mg α-linolenic acid, 200 mg linoleic acid, 110 mg eicosapentenoic acid, and 80 mg docosahexaenoic acid per day; and are permitted their usual other medications. The subjects are assessed before the study, and at intervals during the study, such as every 1, 2, and 4 weeks after the start of a new dose and 4 weeks after the last dose of the combination therapy, for safety and pharmacodynamic evaluations. MRIs of the subjects' livers are taken after 4 weeks at each dose, and 4 weeks after study completion, to determine hepatic fat. At each visit, after a 12-hour fast, blood is drawn and urine collected; and a standard metabolic panel, complete blood count, and standard urinalysis are performed. Blood is analyzed for TC, HDL-C, TG, VLDL-C, LDL-C and apoB. The subjects also maintain health diaries, which are reviewed at each visit.

The combination of MBX-8025 and evolocumab causes dose-dependent lowering of TC, LDL-C, VLDL-C, TG, and apoB, and raising of HDL-C; in particular, increasing the lowering of TC, LDL-C, VLDL-C, TG, and apoB, and raising of HDL-C, beyond that caused by evolocumab alone.

Similar studies may be conducted with MBX-8025 and other PCSK9 inhibitors, such as alirocumab; and an increased reduction in LDL-C over that caused by the PCSK9 inhibitor alone is expectable.

### Example 3

### Dose Escalation Study with MBX-8025 and Evolocumab in Primary Hyperlipidemia with Clinical Atherosclerotic Cardiovascular Disease

Subjects with primary hyperlipidemia and clinical atherosclerotic cardiovascular disease, on maximally-tolerated lipid-lowering therapy and evolocumab at either 140 mg every 2 weeks or 420 mg once monthly, are treated with MBX-8025 L-lysine dihydrate salt at a dose of 50, 100, or 200 mg/day (as MBX-8025 free acid), escalating every 4 weeks. The subjects are permitted their usual other medications. The subjects are assessed before the study, and at intervals during the study, such as every 1, 2, and 4 weeks after the start of a new dose and 4 weeks after the last dose of the combination therapy, for safety and pharmacodynamic evaluations. MRIs of the subjects' livers are taken after 4 weeks at each dose, and 4 weeks after study completion, to determine hepatic fat. At each visit, after a 12-hour fast, blood is drawn and urine collected; and a standard metabolic panel, complete blood count, and standard urinalysis are performed. Blood is analyzed for TC, HDL-C, TG, VLDL-C, LDL-C and apoB. The subjects also maintain health diaries, which are reviewed at each visit.

The combination of MBX-8025 and evolocumab causes dose-dependent lowering of TC, LDL-C, VLDL-C, TG, and apoB, and raising of HDL-C; in particular, increasing the lowering of TC, LDL-C, VLDL-C, TG, and apoB, and raising of HDL-C, beyond that caused by evolocumab alone.

Similar studies may be conducted with MBX-8025 and other PCSK9 inhibitors, such as alirocumab; and an increased reduction in LDL-C over that caused by the other PCSK9 inhibitor alone is expectable.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A method of treating severe hyperlipidemia by administering (R)-2-(4-((2-ethoxy-3-(4-(trifluoromethyl)phenoxy)propyl)thio)-2-methylphenoxy)acetic acid or a salt thereof in combination with a PCSK9 inhibitor.

2. The method of claim 1 where the (R)-2-(4-((2-ethoxy-3-(4-(trifluoromethyl)-phenoxy)propyl)thio)-2-methylphenoxy)acetic acid or a salt thereof is (R)-2-(4-((2-ethoxy-3-(4-(trifluoromethyl)phenoxy)propyl)thio)-2-methylphenoxy)acetic acidL-lysine dihydrate.

3. The method of claim 1 where the dose of (R)-2-(4-((2-ethoxy-3-(4-(trifluoromethyl)phenoxy)propyl)thio)-2-methylphenoxy)acetic acid or a salt thereof (when calculated as the free acid) is 20-200 mg/day.

4.-20. (canceled)

21. The method of claim 3 where the dose of (R)-2-(4-((2-ethoxy-3-(4-(trifluoromethyl)phenoxy)propyl)thio)-2-methylphenoxy)acetic acid or a salt thereof (when calculated as the free acid) is 50-200 mg/day.

22. The method of claim 1 where the(R)-2-(4-((2-ethoxy-3-(4-(trifluoromethyl)-phenoxy)propyl)thio)-2-methylphenoxy)acetic acid or a salt thereof is administered once/day.

23. The method of claim 1 where the PCSK9 inhibitor is evolocumab, alirocumab, bococizumab, RG7652, LGT-209, LY3015014, ALN-PCSsc, or BMS-962476.

24. The method of claim 23 where the PCSK9 inhibitor is evolocumab.

25. The method of claim 23 where the PCSK9 inhibitor is alirocumab.

26. The method of claim 23 where the PCSK9 inhibitor is bococizumab.

27. The method of claim 1 where the severe hyperlipidemia is homozygous familial hypercholesterolemia.

28. The method of claim 1 where the severe hyperlipidemia is heterozygous familial hypercholesterolemia.

29. The method of claim 1 where the severe hyperlipidemia is hyperbetalipoproteinemia.

30. The method of claim 29 where the hyperbetalipoproteinemia is accompanied by atherosclerotic cardiovascular disease.

31. The method of claim 1 where the severe hyperlipidemia is combined hyperlipidemia.

32. The method of claim 31 where the combined hyperlipidemia is accompanied by atherosclerotic cardiovascular disease.

33. The method of claim 1 where a subject suffering from the severe hyperlipidemia fails to achieve adequate control of LDL-C with maximally-tolerated conventional lipid-lowering therapy.

34. The method of claim 1 where a subject suffering from the severe hyperlipidemia fails to achieve adequate control of LDL-C with maximally-tolerated conventional lipid-lowering therapy and therapy with a PCSK9 inhibitor.

35. The method of claim 34 where the subject receiving maximally-tolerated conventional lipid-lowering therapy and therapy with a PCSK9 inhibitor exhibits one or more of:
(a) an absolute reduction in LDL-C of at least 40 mg/dL;
(b) a final LDL-C of not more than 130 mg/dL; and
(c) a percentage reduction in LDL-C of at least 15%,
when receiving both the maximally-tolerated conventional lipid-lowering therapy and therapy with a PCSK9 inhibitor, and therapy with (R)-2-(4-((2-ethoxy-3-(4-(trifluoromethyl)phenoxy)-propyl)thio)-2-methylphenoxy)acetic acid or a salt thereof.

36. The method of claim 35 where the subject receiving maximally-tolerated conventional lipid-lowering therapy and therapy with a PCSK9 inhibitor exhibits one or more of:
(a) an absolute reduction in LDL-C of at least 100 mg/dL;
(b) a final LDL-C of not more than 100 mg/dL; and
(c) a percentage reduction in LDL-C of at least 20%,
when receiving both the maximally-tolerated conventional lipid-lowering therapy and therapy with a PCSK9 inhibitor, and therapy with (R)-2-(4-((2-ethoxy-3-(4-(trifluoromethyl)phenoxy)-propyl)thio)-2-methylphenoxy)acetic acid or a salt thereof.

37. The method of claim 36 where the subject receiving maximally-tolerated conventional lipid-lowering therapy and therapy with a PCSK9 inhibitor exhibits one or more of:
(a) an absolute reduction in LDL-C of at least 150 mg/dL;
(b) a final LDL-C of not more than 70 mg/dL; and
(c) a percentage reduction in LDL-C of at least 30%,
when receiving both the maximally-tolerated conventional lipid-lowering therapy and therapy with a PCSK9 inhibitor, and therapy with (R)-2-(4-((2-ethoxy-3-(4-(trifluoromethyl)phenoxy)-propyl)thio)-2-methylphenoxy)acetic acid or a salt thereof.
